(19) United States
(12) Patent Application Publication
(10) Pub. No.: US 2026/0248459 A1
(43) Pub. Date: Aug. 27, 2026

(54) APPARATUSES AND METHODS FOR IDENTIFICATION AND TREATMENT OF PATIENTS RESPONSIVE TO ANTIPSYCHOTIC AGENT THERAPY

(71) Applicants: Peter J. SIEKMEIER, Cambridge, MA (US); Steven B. LOWEN, Bedford, MA (US); Joseph T. COYLE, Cambridge, MA (US)

(72) Inventors: Peter J. SIEKMEIER, Cambridge, MA (US); Steven B. LOWEN, Bedford, MA (US); Joseph T. COYLE, Cambridge, MA (US)

(21) Appl. No.: 19/335,863

(22) Filed: Sep. 22, 2025

Related U.S. Application Data
(63) Continuation of application No. 17/640,181, filed on Sep. 3, 2020, now abandoned.
(60) Provisional application No. 62/895,081, filed on Sep. 3, 2019.

(51) Int. Cl. A61B 5/00; A61B 5/372; A61N 1/36; A61N 1/372; G16H 20/10; G16H 50/20

(52) U.S. Cl. CPC A61B 5/7267; A61B 5/372; A61B 5/7264; A61B 5/7282; A61N 1/36082; A61N 1/37247; A61N 1/37282; G16H 20/10; G16H 50/20

(57) ABSTRACT

In some embodiments, a method of treating a patient with an antipsychotic agent can include identifying the patient as an antipsychotic agent responder. The method can further include obtaining an electroencephalogram (EEG) signals from the patient. The method can further include measuring one or more EEG metrics, thereby identifying the patient as an antipsychotic agent responder. If the patient is an antipsychotic agent responder, the method can further include then administering the antipsychotic agent.

### CROSS-REFERENCES TO RELATED APPLICATIONS

This application is a continuation of U.S. patent application Ser. No. 17/640,181, filed on Mar. 3, 2020, which is a national phase filing under 35 U.S.C. § 371 of International Patent Application No. PCT/US2020/049194, filed on Sep. 3, 2020, which claims priority to U.S. Provisional Patent Application No. 62/895,081, filed on Sep. 3, 2019, the contents of each of the which is incorporated by reference herein in its entirety for all purposes.

### TECHNICAL FIELD

The present disclosure relates to the field of treatment for psychiatric disorders.

### BACKGROUND

Treatment of mental disorders such as schizophrenia spectrum or other psychotic disorder often includes use of psychiatric drugs. Known antipsychotics include, canonically, dopamine receptor agonists. Efforts to develop glutamate receptor agonists have generally failed in late stage clinical trials. There remains a need in the art for methods of treatment that include identifying patients that are antipsychotic agent responders.

### SUMMARY

In some embodiments, a method of treating a patient with an antipsychotic agent can include identifying the patient as an antipsychotic agent responder. The method can further include obtaining an electroencephalogram (EEG) signals from the patient. The method can further include measuring one or more EEG metrics, thereby identifying the patient as an antipsychotic agent responder. If the patient is an antipsychotic agent responder, the method can further include then administering the antipsychotic agent. In some embodiments, measuring is performed pre-treatment.

In some embodiments, the antipsychotic agent is a glutamate receptor agonist. In some embodiments, the antipsychotic agent is a group II metabotropic glutamate receptor (mGluR2/3) agonist. In some embodiments, the mGluR2/3 agonist is pomaglumetad or a pharmaceutically acceptable salt thereof. In some embodiments, the mGluR2/3 agonist is pomaglumetad methionil or a pharmaceutically acceptable salt thereof.

In some embodiments, the one or more EEG metrics include one or more electrophysiological behaviors at one or more brain locations. In some embodiments, the one or more EEG metrics include one or more electrophysiological behaviors at one or more brain locations under stimulation of the subject. In some embodiments, the stimulation is a photic stimulation, an electrical stimulation, a magnetic stimulation, haptic stimulation, or an acoustic stimulation.

In some embodiments, the electrophysiological behavior under stimulation is selected from:

| | | Predetermined |
|---|---|---|
| Brain Location | EEG Metric | Frequency |
| center frontal | Frequency domain transform | low gamma (30 Hz) |
| left temporal | Frequency domain transform | low gamma (30 Hz) |
| right central | Frequency domain transform | low gamma (30 Hz) |
| center parietal | Frequency domain transform | low gamma (30 Hz) |
| right parietal | Frequency domain transform | low gamma (30 Hz) |
| right rear temporal | Frequency domain transform | low gamma (30 Hz) |
| left occipital | Frequency domain transform | low gamma (30 Hz) |
| right occipital | Frequency domain transform | low gamma (30 Hz) |
| left temporal | Frequency domain transform | low beta (15 Hz) |
| left rear temporal | Frequency domain transform | low beta (15 Hz) |
| left parietal | Frequency domain transform | low beta (15 Hz) |
| center parietal | Frequency domain transform | low beta (15 Hz) |
| left occipital | Frequency domain transform | low beta (15 Hz) |

In some embodiments, the one or more EEG metrics include one or more electrophysiological behaviors in resting state at one or more brain locations, the electrophysiological behavior at the brain location selected from:

| | | Predetermined |
|---|---|---|
| Brain Location | EEG Metric | Frequency |
| left frontal | power law exponent | — |
| center frontal | power law exponent | — |
| right frontal | power law exponent | — |
| left central | power law exponent | — |
| right frontal | power law exponent | — |
| left temporal | power law exponent | — |
| right parietal | power law exponent | — |
| right rear temporal | power law exponent | — |
| left temporal | Frequency domain transform | beta (22 Hz) |
| right central | Frequency domain transform | beta (16-25 Hz) |

In some embodiments, each clinical treatment outcome from the set of clinical treatment outcomes is classified as responsive and non-responsive based on a threshold value or a receiver operating characteristic (ROC) curve.

In some embodiments, the identifying step is performed by a non-transitory processor-readable medium storing code representing instructions to be executed by a processor. The code include code to cause the processor to receive the EEG signals recorded from the one or more brain locations of the patient. The code can include code to cause the processor to transform the EEG signals into the one or more EEG metrics. The code can include further code to cause the processor to execute a model configured to receive the EEG metrics and identify the patient as a antipsychotic agent responder.

In some embodiments, the model is a machine learning model. The code can include further code to cause the processor to train the machine learning model based on a training set including a set of EEG metrics and a set of clinical treatment outcomes associated with the set of EEG metrics.

In some embodiments, each clinical treatment outcome from the set of clinical treatment outcomes is determined based on at least one of the MATRICS™ Consensus Cognitive Battery (MCCB™), a Positive and Negative Syndrome Scale (PANSS) score, and a clinical global impression severity scale (CGI-S).

In some embodiments, the set of clinical treatment outcomes includes a decrease in at least one positive symptom of the PANNSS. In some embodiments, the set of clinical treatment outcomes includes a decrease in at least one negative symptom of the PANNSS. In some embodiments, the antipsychotic agent responder is defined by an increase in working memory performance. In some embodiments, the antipsychotic agent responder is defined by an increase in attention-vigilance. In some embodiments, the antipsychotic agent responder is defined by an increase in reasoning-problem solving.

In some embodiments, the machine learning model includes a feed-forward machine learning model, a convolutional neural network (CNN), a graph neural network (GNN), an auto encoder, or a transformer neural network. In some embodiments, the machine learning model includes a logistic regression model, a Naïve Bayes classifier, a support vector machine (SVM), a random forest, a decision tree, or an extreme gradient boosting (XGBoost) model.

In some embodiments, the EEG metrics include a power law exponent. In some embodiments, the EEG signals being obtained in the delta band, the theta band, the alpha band, the beta band, or the gamma band. In some embodiments, the identifying step identifies the patient as an antipsychotic agent responder using at most 1, at most 2, or at most 3 EEG metrics. In some embodiments, the patient suffers from or is at risk for a psychotic disorder.

### BRIEF DESCRIPTION OF THE DRAWINGS

FIG. 1 is a schematic description of a treatment-response prediction device, according to an embodiment.

FIG. 2 is a flowchart illustrating a method of treatment-response prediction, according to an embodiment.

FIG. 3 is a flowchart illustrating a method of treatment-response prediction, according to an embodiment.

FIG. 4 shows a montage for EEG recording.

FIG. 5 illustrates determining the power law exponent (PLE) of an EEG signal.

FIG. 6 shows a correlation between pre-treatment low-gamma (30 Hz) activity and treatment response based on MCCB attention-vigilance domain score. Patients received photic stimulation at 30 Hz. Coloring indicates correlation coefficient, r, with scale shown at right. Selected r and p values shown in Table 1A.

FIG. 7 shows a correlation between pre-treatment low-beta (15 Hz) activity and treatment response based on MCCB reasoning-problems solving domain score. Patients received photic stimulation at 15 Hz. Coloring indicates correlation coefficient, r, with scale shown at right. Selected r and p values shown in Table 1A.

FIG. 8 shows a correlation between pre-treatment power law exponent and treatment response based on MCCB working memory domain score. EEG readings were taken in resting state. Coloring indicates correlation coefficient, r, with scale shown at right. Selected r values and significance shown in Table 1B.

FIG. 9 shows a receiver operator curve (ROC) for effect shown in FIG. 8, at EEG lead C3. Sensitivity=0.750, specificity=0.897 (area under the curve [AUC]=0.809, p=0.039).

### DETAILED DESCRIPTION

Non-limiting examples of various aspects and variations of the embodiments are described herein and illustrated in the accompanying drawings.

In one aspect, the disclosure provides methods of treating a patient with an antipsychotic agent (e.g., a glutamate receptor agonist). The methods include identifying the patient as an antipsychotic agent responder by obtaining or having obtained electroencephalogram (EEG) signals from the patient. They include measuring or having measuring one or more EEG metrics, thereby identifying the patient as an antipsychotic agent responder, and if the patient is an antipsychotic agent responder, then administering the antipsychotic agent.

In another aspect, one or more embodiments described herein generally relate to apparatus, methods, and systems for dynamically processing structured and semi-structured data, and in particular, apparatus, methods, and systems that use a model (e.g. a neural network model) to efficiently and reliably predict an outcome based on the structured and semi-structured-data. Apparatus, methods and systems of treatment-response prediction are disclosed. In some embodiments, treatment-response can be used to process, for example, EEG signals in form of time series, stationary data, non-stationary-data, linear data, non-linear data, and/or the like.

Described herein are treatment-response prediction apparatuses and methods that predict treatment response based on EEG signals collected from a patient. By enabling identification a patient as an antipsychotic agent responder, prior to treatment, the methods described herein may avoid unnecessary adverse events and side effects of treatment. Moreover, the methods described herein may increase the response rate to the antipsychotic agent. In particular embodiments, the methods described herein enable safe and effective use of the pomaglumetad, pomaglumetad methionil or a pharmaceutically acceptable salt thereof in the treatment of psychiatric disorders (e.g., psychotic disorders). In some embodiments, individual EEG metrics predictive of antipsychotic agent (e.g., glutamate receptor agonist) responder status are disclosed. In some embodiments, responder prediction is improved by training a machine-learning model on multiple EEG metrics.

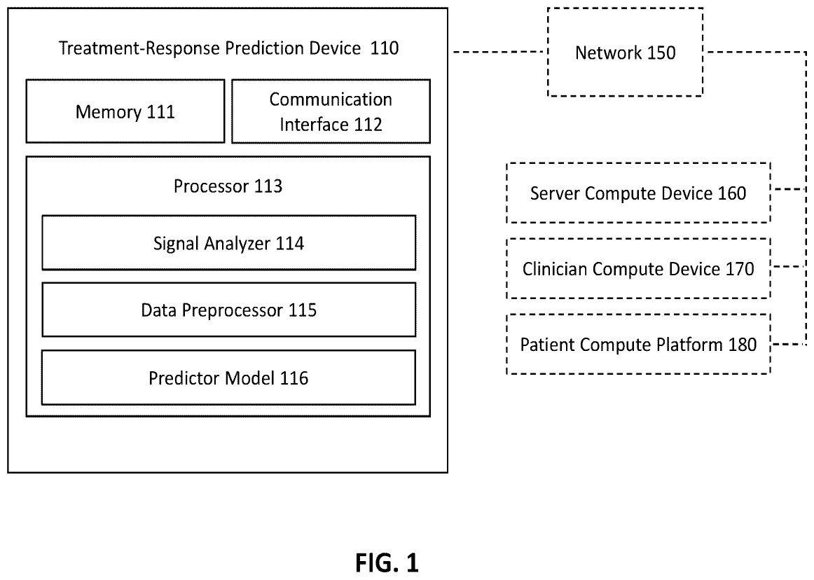

FIG. 1 is a schematic description of a treatment-response prediction device 110, according to an embodiment. The treatment-response prediction device 110 can identify a patient as an antipsychotic agent responder, prior to treatment. The treatment-response prediction device 110 can be also configured to execute a model (e.g., an artificial intelligence model) that predicts a treatment response based on EEG signals collected for a patient. The set of EEG signals are analyzed by the treatment-response prediction device 110 to generate EEG metrics. The treatment-response prediction device 110 can optionally be coupled to a server compute device 160, a clinician programmer device 170, and/or a patient compute device 180 via a network 150. The treatment-response prediction device 110, clinician programmer device 170, and/or a patient compute device 180 each can be a hardware-based computing device and/or a multimedia device, such as, for example, a computer, a desktop, a laptop, a smartphone, a tablet, a wearable device, and/or the like.

The treatment-response prediction device 110 includes a memory 111, a communication interface 112, and a processor 113. The treatment-response prediction device 110 can receive data including EEG signals from an EEG machine (not shown) that records activities of a patient's brain. In some instances, the activities of the patient's brain can be/include electrical activities and the activities can be recorded as EEG signals by a set of electrodes connected to the EEG machine that may be operatively coupled to the treatment-response prediction device 110. The EEG machine can transmit the set of EEG signals to the treatment-response prediction device 110. The EEG signals can be recorded in the memory 111 and analyzed by the processor 113 for treating a patient with a antipsychotic agent.

The network 150 can be a digital telecommunication network of servers and/or compute devices. The servers and/or computes device on the network can be connected via one or more wired or wireless communication networks (not shown) to share resources such as, for example, data storage and/or computing power. The wired or wireless communication networks between servers and/or compute devices of the network 150 can include one or more communication channels, for example, a radio frequency (RF) communication channel(s), an extremely low frequency (ELF) communication channel(s), an ultra-low frequency (ULF) communication channel(s), a low frequency (LF) communication channel(s), a medium frequency (MF) communication channel(s), an ultra-high frequency (UHF) communication channel(s), an extremely high frequency (EHF) communication channel(s), a fiber optic commination channel(s), an electronic communication channel(s), a satellite communication channel(s), and/or the like. The network 150 can be, for example, the Internet, an intranet, a local area network (LAN), a wide area network (WAN), a metropolitan area network (MAN), a worldwide interoperability for microwave access network (WiMAX®), a virtual network, any other suitable communication system and/or a combination of such networks.

The server compute device 160 can be/include compute device mediums specialized for data storage purposes and/or computing purposes that can include, for example, a network of electronic memories, a network of magnetic memories, a server(s), a blade server(s), a storage area network(s), a network attached storage(s), deep learning computing servers, deep learning storage servers, and/or the like. Each server device 160 can include a memory (not shown), a communication interface (not shown) and/or a processor (not shown). The communication interface can receive/transmit data from/to the prediction device 110 via the network 150, the memory can store the data, and the processor can analyze the data. In some instances, the server compute device 160 can be a biobank server that stores the data for a long period of time (e.g. 2 years, 5 years, 10 years, 100 years, and/or the like).

The clinician compute device 170 and/or the patient compute device 180 can be/include compute devices operatively coupled and configured to transmit and/or receive data and/or analytical models to the treatment-response prediction device 110. A user of patient compute device 180 and/or the clinician compute device 170 can use the treatment-response prediction device 110 (partially or fully) for selecting a treatment and/or a treatment-response prediction. In some instances, the patient compute device 180 and/or the clinician compute device 170 can be/include, for example, a personal computer, a laptop, a smartphone, a custom personal assistant device, and/or the like, each including a memory (not shown), a communication interface (not shown) and/or a processor (not shown). The processor of the patient compute device 180 and/or the clinician compute device 170 can include a hardware based integrated circuit (IC) or any other suitable processing device configured to run and/or execute a set of instructions or code. The memory of the patient compute device 180 and/or the clinician compute device 170 can include a hardware based charge storage electronic device or any other suitable data storage medium configured to store data for long term or batch processing of the data by the processor. The communication interface of the patient compute device 180 and/or the clinician compute device 170 can include a hardware based device configured to receive/transmit electric signals, electromagnetic signals, and/or optical signals.

The memory 111 of the treatment-response prediction device 110 can be, for example, a memory buffer, a random access memory (RAM), a read-only memory (ROM), a hard drive, a flash drive, a secure digital (SD) memory card, a compact disk (CD), an external hard drive, an erasable programmable read-only memory (EPROM), an embedded multi-time programmable (MTP) memory, an embedded multi-media card (eMMC), a universal flash storage (UFS) device, and/or the like. The memory 111 can store, for example, one or more software modules and/or code that includes instructions to cause the processor 113 to execute one or more processes or functions (e.g., a signal analyzer 114, a data preprocessor 115, a predictor model 116, and/or the like).

The memory 111 can store a set of files associated with (e.g., generated by executing) the signal analyzer 114, the data preprocessor 115, and/or the predictor model 116. The set of files associated with the signal analyzer 114, the data preprocessor 115, and/or the predictor model 116 can include data generated by the signal analyzer 114, the data preprocessor 115, and/or the predictor model 116 during the operation of the treatment-response prediction device 110. In some instances, the predictor model 116 can be/include a machine learning model. The machine learning model can store temporary variables, return memory addresses, variables, a graph of the machine learning model (e.g., a set of arithmetic operations or a representation of the set of arithmetic operations used by the machine learning model), the graph's metadata, assets (e.g., external files), electronic signatures (e.g., specifying a type of the machine learning model being exported, and the input/output arrays and/or tensors), and/or the like, in the memory 111.

The communication interface 112 of the treatment-response prediction device 110 can include a software component (e.g., executed by processor 113) and/or a hardware component of the treatment-response prediction 110 to facilitate data communication between the treatment-response prediction 110 and external devices (e.g., the server compute device 160, the clinician platform 170, the patient compute device 180, and/or the like) or internal components of the treatment-response prediction 110 (e.g., the memory 111, the processor 113). The communication interface 112 is operatively coupled to and used by the processor 113 and/or the memory 111. The communication interface 112 can be, for example, a network interface card (NIC), a Wi-Fi™ module, a Bluetooth® module, an optical communication module, and/or any other suitable wired and/or wireless communication interface. In some instances, the communication interface 112 can facilitate receiving or transmitting data via the network 150. More specifically, in some implementations, the communication interface 112 can facilitate receiving or transmitting data containing EEG signals, models, and/or the like through the network 150 from/to the server compute device 160, the clinician platform 170, the patient compute device 180, and/or the like, each of which are communicatively coupled to the treatment-response prediction 110 via the network 150. In some instances, the communication interface 112 can facilitate receiving or transmitting data from the EEG machine.

The processor 113 can be, for example, a hardware based integrated circuit (IC) or any other suitable processing device configured to run or execute a set of instructions or a set of code. For example, the processor 113 can include a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC), a graphics processing unit (GPU), a neural network processor (NNP), and/or the like. The processor 113 can be operatively coupled to the memory 111 through a system bus (for example, address bus, data bus, and/or control bus, not shown).

The processor 113 includes a signal analyzer 114, a data preprocessor 115, and a predictor model 116. Each of the signal analyzer 114, the data preprocessor 115, and/or the predictor model 116 can include software stored in the memory 111 and executed by the processor 113. For example, a code to cause the signal analyzer 114 to fetch/process the high dimensional and high volume data can be stored in the memory 111 and executed by the processor 113. Alternatively, each of the signal analyzer 114, the data preprocessor 115, and/or the predictor model 116 can be a hardware-based device. For example, a process to predictor model 116 to predict a clinical outcome can be implemented on an individual integrated circuit chip (e.g., an ASIC).

The signal analyzer 114 can receive the EEG signals and perform signal analysis on the EEG signal. In some instances, the signal analyzer can perform a Fourier analysis (e.g., used for stationary signals) and/or wavelet analysis (e.g., used for non-stationary signals) to transform the EEG signals from time domain to frequency domain. The transformed EEG signals can be further analyzed by the signal analyzer 114 to extract significant frequency components of the EEG signals. Fourier analysis of an EEG signal produces a power spectrum that includes an indication of which frequencies are present in the EEG signals, and relative strength (or power) of the frequencies. Known power spectrum analysis of EEG or magnetoencephalographic (MEG) signals has not revealed particular frequency peaks that robustly differentiates schizophrenic patients from controls.

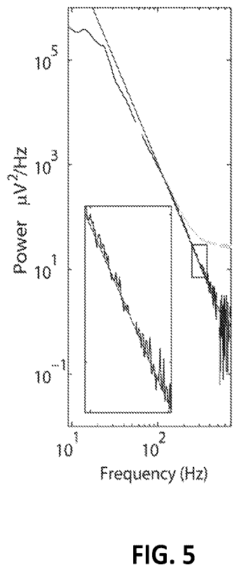

The signal analyzer 114 can further generate EEG metrics. In some instances, the EEG metrics can include a metric that is based on a ratio of an EEG signal power at a first frequency to the EEG signal power at a second frequency (e.g., a ratio of power at 20 Hz/power at 40 Hz power). The first and/or the second frequencies can be picked from a frequency band or multiple frequency bands. In some instances, the EEG metrics can include a power law exponent (also referred to as ‘1/f noise’ or ‘fractal exponent’) of the EEG signals. A power spectrum of EEG signals (at resting state or under stimulation), when viewed in the frequency domain (that is, with frequency of oscillation plotted on the x-axis and power on the y-axis), may be approximated by a straight line, when viewed on a log-log plot (see FIG. 5). Mathematically, the power spectrum can be expressed as log (P)=k−β log (f), or equivalently, P αf−β (=1/fβ), where P represents power, f represents frequency, −β represents the slope of the fitted line, and k represents a constant. The power law exponent, β, is constant (“scale invariant” or “fractal” behavior) regardless of the resolution at which it is calculated (see inset of FIG. 5). Steeper slopes of the power law exponent may reveal a higher degree of “structure” or “memory” in underlying brain interactions.

Oscillatory activity at a number of frequencies and brain locations can be observed in human brain. The EEG signals recorded by the EEG machine can be collected for the frequencies and the brain locations. The brain locations can be based on an EEG electrode map. The frequencies can be in the delta band (1-3 cycles per second [Hz]), the theta band (4-7 Hz), the alpha band (8-12 Hz), the beta band (12-30 Hz), the gamma band (40-80 Hz), and/or the like.

The data preprocessor 115 can be used to receive the data (e.g., including analyzed signals by the signal analyzer 114) and further prepare the data for processing by the predictor model 116. In some implantations, the data preprocessor 115 can normalize the data, feature extraction, dimension reduction, and/or the like. In some instances, normalizing the data may involve amplitude matching, frequency matching, file format (e.g., txt format, CSV format, and/or the like) adjustment, data format (e.g., comma separated, semicolon separated, etc.) adjustment, and/or the like.

In some instances, the data preprocessor 115 can be configured to receive a set of signals, convert a format of the set of signals, remove measurement artifacts (e.g., generated due to eye blinks or scalp muscle movements of a patient from whom the set of signals are taken from), and/or filter the set of signals (e.g., to reduce noise in the set of (denoise) signals). Also, the data preprocessor 115 can be configured to perform an independent component analyses (ICA) to decompose the set of signals into functionally and spatially separated signals.

The predictor model 116 (also referred to as ‘the model’) can be/include a machine learning model, as described in further details herein. The predictor model 116 may include a feed-forward machine learning model, a convolutional neural network (CNN), a graph neural network (GNN), an auto encoder, a transformer neural network, a logistic regression model, a Naive Bayes classifier, a support vector machine (SVM), a random forest, a decision tree, an extreme gradient boosting (XGBoost) model, and/or the like. The predictor model 116 can be configured to include a set of model parameters including a set of weights, a set of biases, and/or a set of activation functions that, once trained, may be executed to generate a prediction of clinical outcome (e.g., responder, non-responder, 20% responder, 99% responder, a response score, and/or the like) of the EEG signals. For example the predictor model 116 can be configured to predict antipsychotic agent treatment responders.

In some implantations, the clinical outcome can be categorized as “responder” vs. “non-responder”. Such categorization can be defined in multiple ways including:
Based on percentage improvement, averaged across all items of the MCCB cognitive battery.
Based on percentage improvement, averaged across all items of the positive symptom scale.
Based on percentage improvement, averaged across all items of the negative symptom scale.
Based on an average of (i), (ii), and (iii) above, as well as percentage change in Clinical Global Impression Severity Scale (CGI-S).

In all the above cases, percentage improvement from baseline will be taken as the outcome measure. “Response” can be defined using a number of different cutoffs (e.g., improvement of 20%, 30%, 40%, 50% and 60%).

In one example, the predictor model 116 can be/include a feed forward neural network or a deep learning model that includes an input layer, an output layer, and multiple hidden layers (e.g., 5 layers, 10 layers, 20 layers, 50 layers, 100 layers, 200 layers, etc.). The multiple hidden layers may include normalization layers, fully connected layers, activation layers, convolutional layers, recurrent layers, and/or any other layers that are suitable for representing a correlation between the EEG signals and the clinical outcome, each score representing an energy term.

In one example, the predictor model 116 can be an XGBoost model that includes a set of hyper-parameters such as, for example, a number of boost rounds that defines the number of boosting rounds or trees in the XGBoost model, maximum depth that defines a maximum number of permitted nodes from a root of a tree of the XGBoost model to a leaf of the tree, and/or the like. The XGBoost model may include a set of trees, a set of nodes, a set of weights, a set of biases, and other parameters useful for describing the XGBoost model.

In some implementations, the predictor model 116 can be configured to iteratively receive EEG signals and/or EEG metrics and generate an output predicting the clinical outcome (e.g., a binary response in which 1 represents responder and 0 represents non-responder). The EEG signals and/or EEG metrics can be associated with one clinical outcome. True clinical outcomes can be compared to outputs from the predictor model 116 using an optimization model and an objective function (also referred to as ‘cost function’) to generate a training loss value. The objective function may include, for example, a mean square error, a mean absolute error, a mean absolute percentage error, a logcosh, a categorical cross entropy, and/or the like. The set of model parameters of the predictor model 116 can be modified in multiple iterations and the first objective function can be executed at each iteration until the training loss value converges to a first predetermined training threshold (e.g. 80%, 85%, 90%, 97%, etc.).

In some embodiments, the predictor model 116 can integrate the EEG metrics and/or EEG signals to generate a composite score that identifies the patient as an antipsychotic agent responder. In some instances, the composite score can be a normalized range of 0 to 100. A threshold within the normalized range can be set to determine whether a subset of EEG metric and/or EEG signals of a patient can identify the patient as an antipsychotic agent responder.

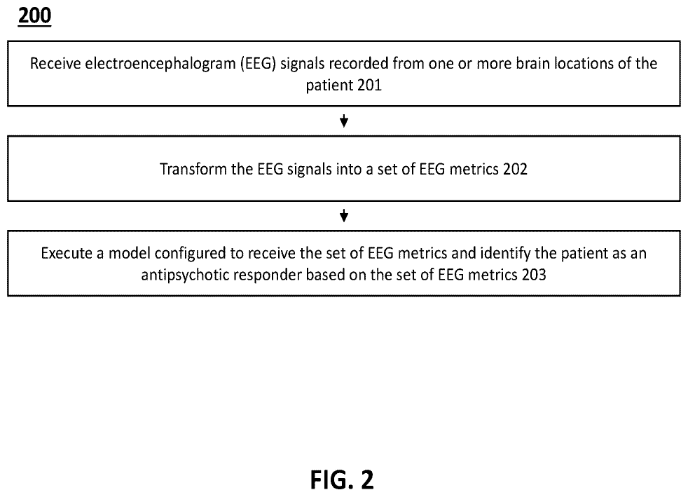

FIG. 2 is a flowchart illustrating a method 200 of treatment-response prediction, according to an embodiment. The method 200 can be performed by a treatment-response prediction device (such as the treatment-response prediction device as shown and described with respect to FIG. 1). The method 200 can include receiving 201 electroencephalogram (EEG) signals recorded from one or more brain locations of the patient. The method 200 can further include transforming 202 the EEG signals into a set of EEG metrics. The EEG metrics can include electrophysiological behaviors under stimulation or in rest at a set of brain locations. The stimulation can include a photic stimulation, an electrical stimulation, a magnetic stimulation, haptic stimulation, and/or an acoustic stimulation. The method 200 can further include executing 203 a model to receive the set of EEG metrics and identify the patient as an antipsychotic agent responder based on the set of EEG metrics. In some implementations, the model is a machine leaning model.

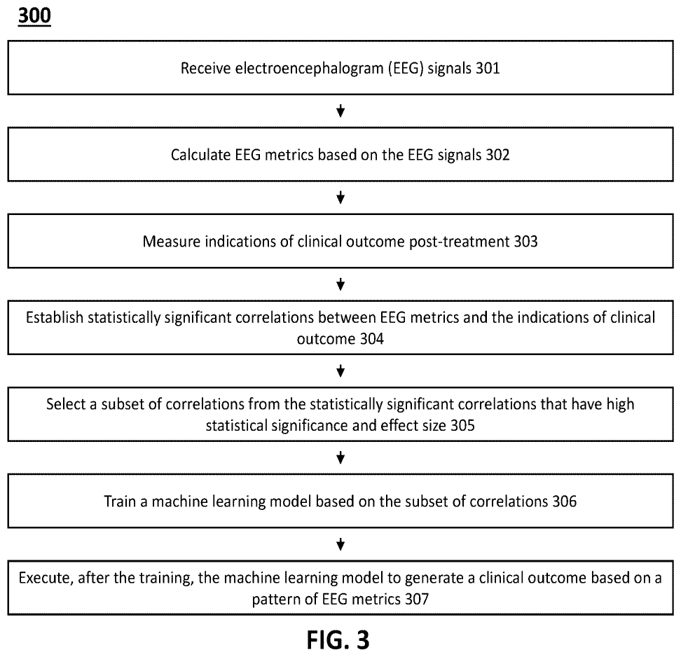

FIG. 3 is a flowchart illustrating a method 300 of treatment-response prediction, according to an embodiment. The method 300 can be performed by a treatment-response prediction device (such as the treatment-response prediction device as shown and described with respect to FIG. 1). The method 300 can include receiving 301 electroencephalogram (EEG) signals (e.g., for a set of electrodes). In some instances, the EEG signals can be measured pretreatment. In some instances, the EEG signals can be analyzed to calculate power in one or more frequency range (also termed a power band) (e.g., delta band, 1-4 Hz; gamma band (30-80 Hz)), and/or fractal exponents (power law exponent) of the EEG signals. The method 300 can further include determining 302 a set of EEG metrics; each EEG metric can be identified/generated based on (a) power at a particular frequency band (e.g., the delta band, the beta band, the gamma band, and/or the like), (b) a ratio of the power of two different frequency bands (e.g., a first power at the beta band/a second power at the gamma band), (c) the power law exponent, and/or (d) power of the EEG signal at a driven frequency (i.e., sensory stimulation frequency). The method 300 can further include measuring 303 indications of clinical outcome post-treatment. In some instances, the indications of clinical outcome can be determined by a clinician. In some instances, the indications of clinical outcome can be determined by the treatment-response prediction device based on a set of medical readings.

The method 300 can further include establishing 304 statistically significant correlations between EEG metrics and the indications of clinical outcome. In some instances statistical significance can be calculated and/or represented by p-value as shown, for example, in Table 1A and Table 1B). In some instances, a principal component analysis (PCA) can be carried out to arrive at a set of principal components (PCs) that include 75% variance of independent variables (e.g., pre-treatment EEG metric). In some instances, a multivariate analysis of covariance (MANCOVA) can be carried out with all clinical outcomes as dependent variables, and independent factors of (i) treatment class, indicating treatment with pomaglumetad vs. placebo; (ii) principal components (PC); and (iii) interaction between (i) and (ii). This determines whether or not there was a relationship between any principal component and any clinical response and, more importantly, if there was a significant interaction—which would indicate a difference between treated and non-treated subjects. A significance level of p<0.05 can be used.

In some instances, for PCs that showed significant interactions above, an analysis of covariance (ANCOVA) can be conducted to determine whether there was a significant relationship (e.g., p<0.05) between the PC and any number of clinical outcome measures, for treated patients. For those clinical outcome measures identified in step above, correlation between that outcome measure and the independent variables (e.g., a particular EEG metric at a particular EEG electrode) for that sub-analysis can be determined. All independent variables that showed a correlation with the outcome measure at a significance level of p<0.05 can be considered for analysis. These can be ranked from lowest (most significant) to highest, and the relationship(s) showing the highest level of significance for each sub-analysis can be examined in more details by constructing a topographic cortical map including electrode-level correlation and significance levels for that independent variable-dependent variable (that is, EEG metric-clinical outcome measure) pair.

The method 300 can further include selecting 305 a subset of correlations from the statistically significant correlations that have high statistical significance and effect size (e.g., large correlation coefficient). The method 300 can further include training 306 a machine learning model based on the subset of correlations. The method 300 can further include executing 307, after the training, the machine learning model to generate a clinical outcome based on a pattern of EEG metrics.

In one aspect, the disclosure provides methods of treating a patient with a antipsychotic agent. The methods include identifying the patient as an antipsychotic agent (e.g., glutamate receptor agonist) responder by obtaining or having obtained a electroencephalogram (EEG) signals from the patient. They include measuring or having measuring one or more EEG metrics, thereby identifying the patient as an antipsychotic agent responder, and if the patient is an antipsychotic agent responder, then administering the antipsychotic agent.

As used herein, the term “patient” refers to a human subject suffering from or at risk for a psychiatric disorder. The patient presents one or more symptoms of a mental disorder. Illustrative psychiatric disorders are described in the Diagnostic and Statistical Manual of Mental Disorders (DSM-5) (5^{th} ed.) (2013), which is incorporated by reference for the purpose of defining mental disorders and symptoms by which patients can be identified as suffering from or at risk for a mental disorder. In some embodiments, the mental disorder is schizophrenia spectrum or other psychotic disorder.

According to the methods of the disclosure, a patient may be identified as a likely responder to an antipsychotic agent (e.g., glutamate receptor agonist). Other factors, including symptoms of the mental disorder, may be considered by the treating physician or other healthcare worker. The methods of the disclosure are not limited to schizophrenia spectrum or other psychotic disorders, as it will be understood that antipsychotic agent may be used to treat other disorders. Without being bound by theory, it is believed that the EEG metrics disclosed herein are predictive of response due the correlation between EEG signals and the underlying biochemistry of the human brain. Features of the brain physiology underlying responsiveness to treatment in psychotic patients may extend to other disorders, including neurodevelopment disorders, bipolar and related disorders, depressive disorders, anxiety disorders, and others.

Given the observed prediction of response to treatment in particular clinical domains (attention-vigilance, reasoning-problem solving, and working memory), the methods of the disclosure may be applied to mental disorders that affect these clinical domains, including but not limited to schizophrenia spectrum and other psychotic disorders.

Illustrative antipsychotics (known in the art as “typical antipsychotics”) that can be administered according to the method of the disclosure or for which responders can be identified include, without limitations: Chlorpromazine, Fluphenazine, Haloperidol, Loxapine, Perphenazine, Pimozide. Thioridazine, Thiothixene, Trifluoperazine.

Further illustrative antipsychotic drugs (known in the art as “atypical antipsychotics”) that can be administered according to the method of the disclosure or for which responders can be identified include, without limitations: aripiprazole (marketed as Abilify), asenapine (marketed as Saphris), clozapine (marketed as Clozaril), iloperidone (marketed as Fanapt), lurasidone (marketed as Latuda), olanzapine (marketed as Zyprexa), olanzapine/fluoxetine (marketed as Symbyax), paliperidone (marketed as Invega), paliperidone, pimavanser, quetiapine (marketed as Seroquel), risperidone (marketed as Risperdal), ziprasidone (marketed as Geodon), or derivatives thereof.

Glutamate receptor agonists have to date failed to achieve the clinical utility expected from preclinical studies. The methods of the disclosure, with associated computational methods and apparatuses, provide for successful treatment with glutamate receptor agonists that otherwise could not be safe and effective.

Illustrative glutamate receptor agonists include D-serine, CTP-692 (deuterated D-serine), SAGE-718 (positive allosteric modulator [PAM] at the NMDA receptor), sarcosine (GlyT-1 inhibitor; also increases glycine), LY379268, eglumegad, pomaglumetad (LY2140023), and pomaglumetad methionil, or pharmaceutically acceptable salts thereof.

In some embodiments, the glutamate receptor agonist is D-serine or a pharmaceutically acceptable salt thereof.

In some embodiments, the glutamate receptor agonist is CTP-692 or a pharmaceutically acceptable salt thereof.

In some embodiments, the glutamate receptor agonist is SAGE-718 or a pharmaceutically acceptable salt thereof.

In some embodiments, the glutamate receptor agonist is a GlyT-1 inhibitor or a pharmaceutically acceptable salt thereof. In some embodiments, the glutamate receptor agonist is a bifoperfin, PF-3463275,GSK1018921, Org25935, AMG747, SSR504734, SSR103800, DCCCyB, R231857, R213129. ASP2535, or a derivative of any of the foregoing, or a pharmaceutically acceptable salt thereof. The chemical structures of these molecules are provided below:

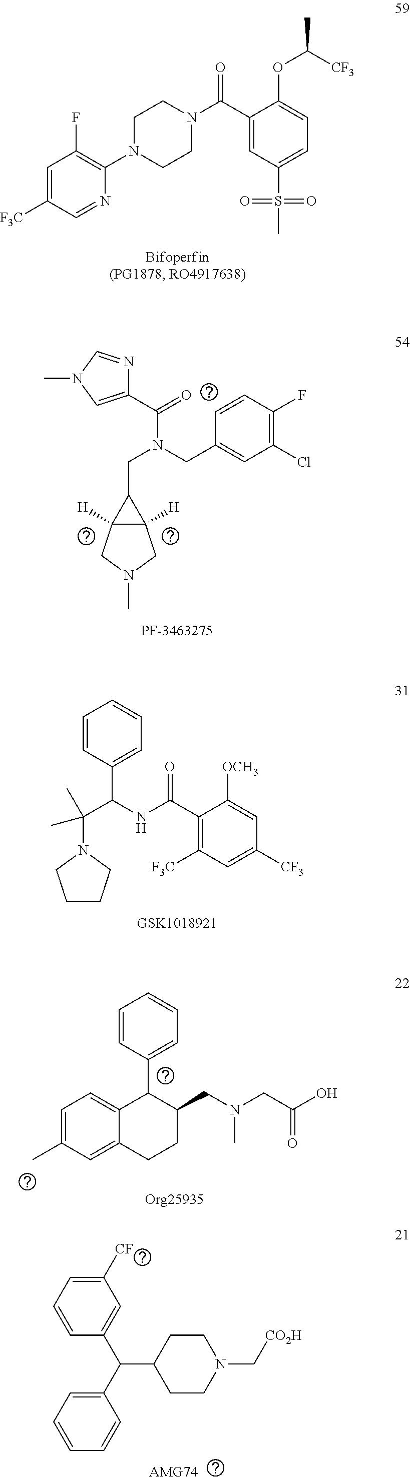

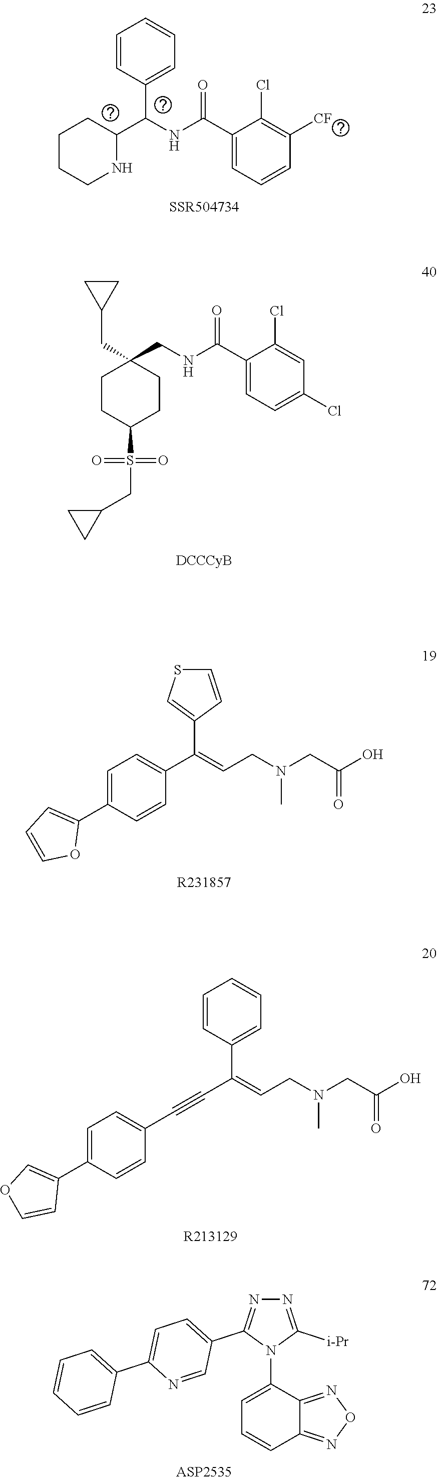

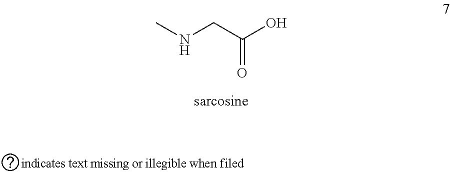

In some embodiments, the glutamate receptor agonist is sarcosine or a pharmaceutically acceptable salt thereof. Sarcosine is a GlyT-1 inhibitor; it also increases glycine.

In some embodiments, the glutamate receptor agonist is LY379268 or a pharmaceutically acceptable salt thereof.

In some embodiments, the glutamate receptor agonist is eglumegad or a pharmaceutically acceptable salt thereof.

In some embodiments, the glutamate receptor agonist is pomaglumetad or a pharmaceutically acceptable salt thereof.

In some embodiments, the glutamate receptor agonist is pomaglumetad methionil or a pharmaceutically acceptable salt thereof.

Pomaglumetad is an amino acid analog drug that acts as a highly selective agonist for the metabotropic glutamate receptor group II subtypes mGluR2 and mGluR3. Human studies investigating therapeutic use of pomaglumetad have focused on the prodrug pomaglumetad methionil, since pomaglumetad exhibits low oral absorption and bioavailability in humans. The dosage of pomaglumetad methionil given to patients has varied by clinical trial, though dosages have typically ranged between 10 mg and 40 mg twice daily (BID). In an early phase II monotherapy trial, the dosage shown to be efficacious was 40 mg BID. Later trials investigating the use of pomaglumetad methionil as an adjuvant to the antipsychotic agent medications already used by patients participating in the study utilized a lower dose of 20 mg BID. If treatment was well tolerated after a week at this target dose, the dose was increased to 40 mg BID. However, if the 20 mg dose was not well tolerated, the dose was decreased to 10 mg.

Clinical studies of pomaglumetad methionil were halted because the glutamate receptor agonist was not significantly more efficacious than the placebo as determined with PANSS total scores. The apparatuses and methods of the present disclosure relate to the surprising discovery that EEG metrics, alone or combined using machine learning-based models, can select patients that respond to glutamate receptor agonist (e.g., pomaglumetad methionil) therapy and/or can predict clinical outcome based on the EEG metrics.

Depending on the specific conditions being treated, such agents may be formulated into liquid (e.g., solutions, suspensions, or emulsions) or solid dosage forms (capsules or tablets) and administered systemically or locally. The agents may be delivered, for example, in a timed-, controlled, or sustained-slow release form as is known to those skilled in the art. Techniques for formulation and administration may be found in Remington: The Science and Practice of Pharmacy (20 ^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery. In some embodiments, the pharmaceutical composition is administered orally. In some embodiments, the pharmaceutical composition is administered intravenously. In some embodiments, the pharmaceutical composition is administered intramuscularly. In some embodiments, the pharmaceutical composition is administered intrathecally. In some embodiments, the pharmaceutical composition is administered subcutaneously.

The pharmaceutical composition or combination of the present invention can be in a unit dosage form (e.g., tablet, capsule, caplet or particulate), wherein the appropriate dosage of the active ingredient may vary depending upon a variety of factors, such as, for example, the age, weight, sex, the route of administration or salt employed.

In general, the presently disclosed methods of treatment result in a decrease in the severity of a disease or condition in a subject. The term “decrease” is meant to inhibit, suppress, attenuate, diminish, arrest, or stabilize a symptom of a disease or condition.

The term “treat” “treating” “treatment” or “therapy”, as used herein, means obtaining beneficial or desired results, for example, clinical results. Beneficial or desired results can include, but are not limited to, alleviation of one or more symptoms of schizophrenia, as defined herein, such as positive symptoms of schizophrenia or negative symptoms of schizophrenia, as herein defined. One aspect of the treatment is, for example, that said treatment should have a minimal adverse effect on the patient, e.g., it should have a high level of safety. The term “alleviation”, for example in reference to a symptom of a condition, as used herein, refers to reducing at least one of the frequency and amplitude of a symptom of a condition in a patient. In one embodiment, the term “method for the treatment”, as used herein, refers to “method to treat”.

In some embodiments of the methods of the disclosure, the antipsychotic agent (e.g. glutamate receptor agonist) is administered in an amount effective to cause the desired therapeutic effect (i.e., in a therapeutically effective amount).

The term “antipsychotic”, as used herein, refers to a neuroleptic drug used to treat a psychotic disorder, such as schizophrenia. In one embodiment, the antipsychotic is, for example, selected from the group comprising a typical antipsychotic and an atypical antipsychotic. In another embodiment, the antipsychotic is a typical antipsychotic. In yet another embodiment, the antipsychotic is an atypical antipsychotic.

The term “typical antipsychotic”, as used herein, refers to a first-generation antipsychotic, for example selected from the group comprising a butyrophenone (e.g., haloperidol), a diphenylbutylpiperidine (e.g., pimozide), a phenothiazine (e.g., chlorpromazine, fluphenazine, perphenazine, prochlorperazine, trifluoperazine), and a thioxanthene (e.g., thiothixene). In one embodiment, the typical antipsychotic is selected from the group comprising haloperidol, pimozide, chlorpromazine, fluphenazine, perphenazine, prochlorperazine, trifluoperazine, and thiothixene; or salts thereof.

The term “atypical antipsychotic”, as used herein, refers to a second-generation antipsychotic, for example selected from the group comprising a benzamide (e.g., sultopride), a benzisoxazole/benzisothiazole (e.g., lurasidone, paliperidone, risperidone), a phenylpiperazine/quinolinone (e.g., aripiprazole, brexpiprazole, cariprazine) a tricyclic (e.g., clozapine, olanzapine, quetiapine, asenapine, zotepine). In one embodiment, the atypical antipsychotic is selected from the group comprising sultopride, lurasidone, paliperidone, risperidone, brexpiprazole, cariprazine, clozapine, olanzapine, quetiapine, asenapine and zotepine; or salts thereof.

### EXAMPLES

The following specific examples are illustrative and do not limit the scope of the invention as claimed.

### Methods

### Preprocessing

After receiving the data, the format of the data can be converted (e.g., change of file format). The data can be further processed to remove measurement artifacts (e.g., due to eye blinks or scalp muscle movements), and filter it. In addition or alternatively, independent component analyses (ICA) can be performed. ICA is a process that allows one to decompose the recorded EEG data into functionally and spatially separated signals (Onton et al. (2006) Neurosci Biobehav Rev 30 (6): 808-822.); this can also serve to denoise the signals. The analytic techniques that follow can be applied to the filtered, artifact-free data, as well as the estimated sources as revealed by ICA.

### Power Spectrum Analysis

Oscillatory activity at a number of different frequencies has been observed in human brain. These are conventionally divided into the slow frequency ranges of delta (1-3 cycles per second [Hz]) and theta (4-7 Hz); the intermediate frequencies, alpha (8-12 Hz) and beta (12-30 Hz); and the gamma band (40-80 Hz).

Fourier analysis of an EEG signal produces a so-called power spectrum—that is, an indication of which frequencies are present, and their relative strength (or power). To our knowledge, this power spectrum analysis has never successfully been used to predict medication response, or to subcategorize schizophrenic patients.

In some instances, wavelet analysis can be used to analyze the EEG signal. The wavelet analysis is a methodology that provides similar information, but uses a rolling time window to determine frequencies present. This is particularly well suited for sets of shorter (<10 sec) data segments that can result after artifact removal.

### Power-Law Behavior

The power spectrum of resting state EEG signals, when viewed in the frequency domain (that is, with frequency of oscillation on the x-axis and power on the y-axis), forms roughly a straight line, when viewed on a log-log plot; see FIG. 5 (from Miller et al. (2019) PLOS Comput Biol 5 (12): e1000609)

Mathematically, this can be expressed as log (P)=k−β log (f), or equivalently, P α f−β (=1/f β), where P=power, f=frequency, −β is the slope of the fitted line, and k is a constant. This phenomenon is known by various terms, such as a “power law” spectrum or “1/f noise”. It is often referred to as “scale invariant” or “fractal” behavior (Lowen and Teich, 2005), as the power law exponent, β, is constant regardless of the resolution at which it is calculated.

### Treatment Response

Response to treatment is determined by calculating change from baseline in key clinical outcome measures, as follows:

Positive and Negative Symptom Scale (PANSS). Correlations are made up of the Positive subscale (which ranges from 7 to 49), the Negative subscale (7 to 49) the general psychopathology subscale (16 to 112), and the PANNS total score (30 to 210).

Clinical Global Impression Severity Scale (CGI-S). This measures overall severity of patients' symptoms and ranges from 1 to 7.

16-Item Negative Symptoms Assessment (NSA-16). Total score, ranging from 16 to 96, is used.

Measurement and Treatment Research to Improve Cognition in Schizophrenia Consensus Cognitive Battery (MCCB). This assesses cognitive functioning in 7 domains (speed of processing, working memory, verbal leaning, visual learning, reasoning and problem solving, attention/vigilance, and social cognition).

Personal and Social Performance (PSP) scale. Total score, which ranges to 100 and assesses four domains of functioning, is used.

### Example 1

Pomaglumetad methionil (LY-2140023, or “poma”) is an experimental antipsychotic agent drug which is an agonist at metabotropic (mGluR2/3) glutamate receptors, and has no known effects on dopamine receptors. This profile differs from all currently used antipsychotic agents, which act on the dopamine (DA) system. Multiple phase II and III clinical trials suggested that while this agent may not be effective for patients with schizophrenia as a whole, there may be particular subgroups for whom it is uniquely helpful. Our objective was to develop novel EEG biomarkers to identify patients with schizophrenia who are more likely to show a positive response to treatment with poma. Previous attempts to use EEG readouts to predict antipsychotic agent treatment responders have largely been unsuccessful. These studies generally studied DA acting antipsychotic agents. Furthermore, we examined additional EEG measures—e.g., response to photic stimulation, magnitude of power law exponent (PLE)—that, to our knowledge, have not been used in prior predictive studies.

### Methods

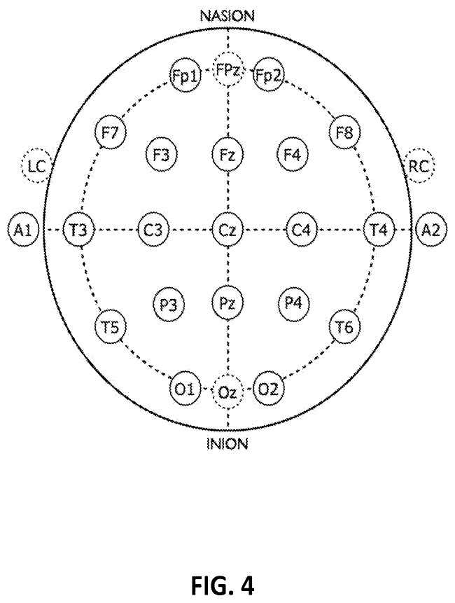

This study used data from clinical trials NCT00845026 (N=117) and NCT01052103 (N=196), which studied male and female patients with schizophrenia treated with poma vs. antipsychotic agent standard of care. EEG recordings were taken in the pre-treatment period using a standard 19-lead montage (FIG. 4), both in the resting state and when patients were exposed to photic stimulation (flashing light), at frequencies ranging from 1 to 30 Hz. We subjected resting EEG data to known power spectrum analysis to determine strength of activity in the delta (1-4 Hz), theta (4-7 Hz), alpha (8-13 Hz), beta (13-30 Hz), and gamma (30-80 Hz) frequency bands. We calculated only power at the stimulated frequency and, for recordings in the resting state, the power law exponent (PLE; also known as the “fractal exponent”). The power spectrum of resting state EEG signals, when viewed in the frequency domain—that is, with power graphed as a function of oscillatory frequency—often forms a roughly straight line when viewed on a log-log plot; the slope of the fitted line is the PLE (see FIG. 5 for example). We calculated all predictive measures for each EEG electrode.

Response to poma treatment was operationalized as percentage change from baseline to trial endpoint on several clinical outcome measures, including the Positive and Negative Symptom Scale (PANSS), and the MATRICS Consensus Cognitive Battery (MCCB) and its seven individual cognitive domain scales. We performed statistical analysis to determine whether there was a significant relationship between any of our calculated EEG metrics in the pre-treatment condition and treatment outcomes. The positive symptom of the PANNSS can include delusions, conceptual disorganization, hallucinations, excitement, grandiosity, suspiciousness/persecution, hostility, and/or the like. The negative symptom of the PANNSS can include blunted affect, emotional withdrawal, poor rapport, passive/apathetic social withdrawal, difficulty in abstract thinking, lack of spontaneity and flow of conversation, stereotyped thinking, and/or the like.

### Results

Surprisingly, we identified a number of pre-treatment EEG metrics that correlated with clinical outcomes and predict response to treatment with poma (Table 1A and Table 1B).

TABLE 1A
Photic Stimulation

| | EEG | | | | | | |
|---|---|---|---|---|---|---|---|
| | Lead | Brain Location | Electrophys Behavior | Clinical Domain | FIG. | r | p-value |
| 1 | Fz | center frontal | low gamma (30 Hz) | attention-vigilance | 6 | 0.319 | 0.000042 |
| 2 | T3 | left temporal | low gamma (30 Hz) | attention-vigilance | 6 | 0.339 | 0.000013 |
| 3 | C4 | right central | low gamma (30 Hz) | attention-vigilance | 6 | 0.313 | 0.000060 |
| 4 | Pz | center parietal | low gamma (30 Hz) | attention-vigilance | 6 | 0.370 | 0.000002 |
| 5 | P4 | right parietal | low gamma (30 Hz) | attention-vigilance | 6 | 0.374 | 0.000001 |
| 6 | T6 | right rear temporal | low gamma (30 Hz) | attention-vigilance | 6 | 0.385 | 0.000001 |
| 7 | O1 | left occipital | low gamma (30 Hz) | attention-vigilance | 6 | 0.342 | 0.000010 |
| 8 | O2 | right occipital | low gamma (30 Hz) | attention-vigilance | 6 | 0.361 | 0.000003 |
| 9 | T3 | left temporal | low beta (15 Hz) | reasoning-problem solving | 6 | 0.196 | 0.024550 |
| 10 | T5 | left rear temporal | low beta (15 Hz) | reasoning-problem solving | 7 | 0.244 | 0.004932 |
| 11 | P3 | left parietal | low beta (15 Hz) | reasoning-problem solving | 7 | 0.217 | 0.012777 |
| 12 | Pz | center parietal | low beta (15 Hz) | reasoning-problem solving | 7 | 0.181 | 0.038468 |
| 13 | O1 | left occipital | low beta (15 Hz) | reasoning-problem solving | 7 | 0.210 | 0.016154 |

TABLE 1B
Resting State

| | EEG | | | | | | |
|---|---|---|---|---|---|---|---|
| | Lead | Brain Location | Electrophys Behavior | Clinical Domain | FIG. | r | p-value |
| 14 | F3 | left frontal | power law exponent | working memory | 8 | 0.275 | 0.000996 |
| 15 | Fz | center frontal | power law exponent | working memory | 8 | 0.253 | 0.002556 |
| 16 | F4 | right frontal | power law exponent | working memory | 8 | 0.309 | 0.000204 |
| 17 | C3 | left central | power law exponent | working memory | 8 | 0.288 | 0.000558 |
| 18 | C4 | right frontal | power law exponent | working memory | 8 | 0.261 | 0.001837 |
| 19 | T5 | left temporal | power law exponent | working memory | 8 | 0.266 | 0.001462 |
| 20 | P4 | right parietal | power law exponent | working memory | 8 | 0.299 | 0.000330 |
| 21 | T6 | right rear temporal | power law exponent | working memory | 8 | 0.297 | 0.000372 |
| 22 | T3 | left temporal | beta (22 Hz) | attention-vigilance | — | 0.222 | 0.003470 |
| 23 | C4 | right central | beta (16-25 Hz) | reasoning-problem solving | — | 0.198 | 0.020872 |

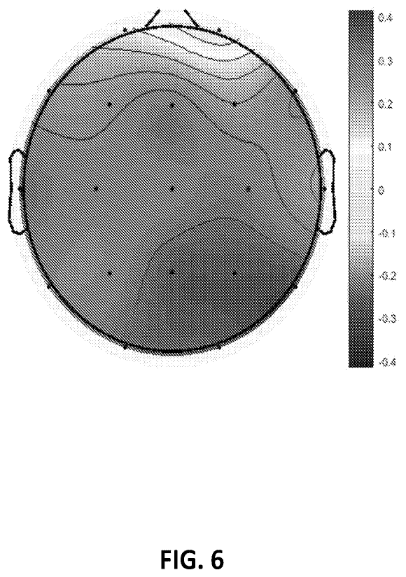

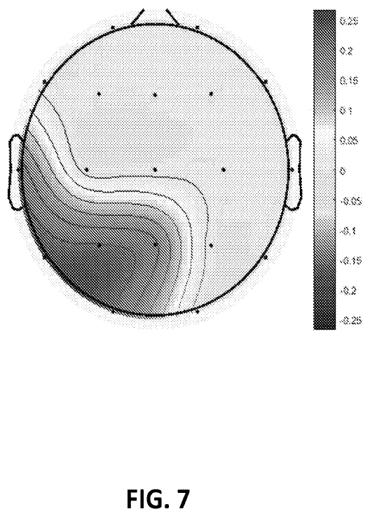

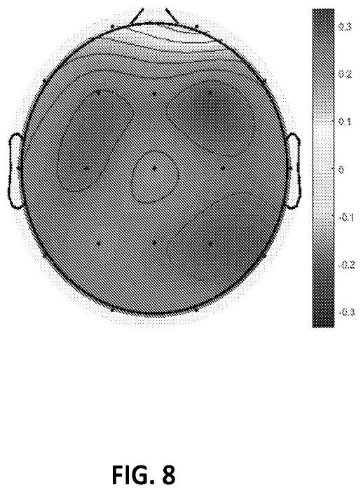

For example: (1) In the 30 Hz photically stimulated condition, there was a positive correlation between pre-treatment low gamma (30 Hz) activity in EEG lead T6 and post-treatment improvement in cognition, as measured by the attention-vigilance domain score of the MCCB (r=0.385, p=0.000001) (line 6 of Table 1A). Correlation coefficients ranged from 0.34 to 0.39 with similar levels of significance at other occipital and inferior leads (FIG. 6). (2) In the 15 Hz photically stimulated condition, there was a positive correlation between pre-treatment low beta (15 Hz) activity at EEG lead T5 and post-treatment improvement in cognition, as measured by the reasoning-problem solving domain score of the MCCB (r=0.244, p=0.004932) (line 10 of Table 1A). Correlation coefficients ranged from 0.19 to 0.24, with similar significance levels, in other left tempo-parietal leads (FIG. 7). (3) For left central electrode C3, there was a positive correlation between pre-treatment PLE and improvement in the working memory (WM) domain score of the MCCB (r=0.288, p=0.000558) (line 17 of Table 1B). Correlation coefficients of 0.25 to 0.31, with similar significance levels, were seen at other fronto-central leads (FIG. 8).

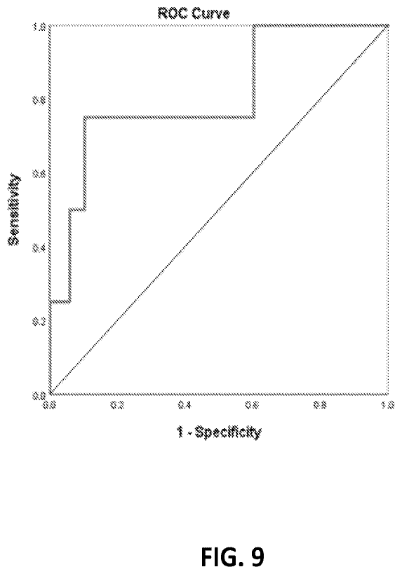

Taking this effect (pre-treatment PLE at left central electrode C3) as an example and using improvement in WM performance of 50% as the definition of treatment response, receiver operator curve (ROC) analysis revealed that PLE at lead C3 could identify poma responders with a sensitivity of 0.750 and a specificity of 0.897 (AUC=0.809, p=0.039) (FIG. 9). In all, 23 lead-level relationships were judged to have relatively high effect size and robust statistical significance, and thus potential clinical utility (Table 1A and Table 1B). Notably, all response variables that we identified involved improvement in cognitive functioning, rather than positive or negative symptoms or other measures of psychopathology. The positive EEG predictors did not occur in one particular cortical area.

In sum, this data demonstrates there are patient subgroup(s) which shows unique benefit in terms of cognitive improvement, and that may be characterized by particular EEG “spectral fingerprints.”

### Example 2

We have identified individual EEG-based pre-treatment metrics that serve as a priori biomarkers of treatment outcome. The methodology described here could be applied to other psychoactive medications, beyond poma, and for a variety of conditions aside from schizophrenia, as these also may result in unique patterns of EEG activity in responders vs. non-responders that may be difficult to appreciate with known methods alone. In treatment settings, the ability to pinpoint likely medication responders, and therefore decrease time and resources devoted to pharmacologic trial and error would have clear value. Also, markers of the kind we have developed could be used to enrich patient samples for clinical trials, potentially resulting in smaller studies and shorter trials, and overall lower drug development costs.

### Example 3

The particular brain phenotype that is uniquely responsive to poma is characterized by a combination of the effects described in Examples 1 and 2. We have developed an artificial intelligence (AI) approach using deep learning artificial neural nets (ANNs) to identify such patterns. This methodology is well-suited to complex, non-linear, pattern recognition tasks with multiple inputs. The resultant “composite biomarker” takes into account all of the effects identified, and is a more robust predictor than any one singly.

We propose to create a deep neural network consisting of four layers: an input layer of 23 nodes, each corresponding to one of the biomarkers of Table 1; two hidden layers—hidden layer one consisting of 33 nodes and the hidden layer two consisting of 7 nodes—and an output layer consisting of one node, representing “responder” when active, and “non-responder” when inactive. The model can be a four layer model and number of nodes in the first and second hidden layers layer can be:

$$\sqrt{( m + 2 ) N} + 2 \sqrt{N / ( m + 1 )} and m \sqrt{N / ( m + 2 )}$$

nodes, respectively, where m is the number of output neurons, and N is the number of samples to be learned. The transfer function to be used will be the so-called rectified linear unit (ReLU), (f(z)=max(z, 0)).

Achieving optimal network architecture (e.g., number of layers, number of nodes per layer) can be approached as an optimization challenge. A number of different methodologies have been suggested to address this problem (Thomas and Suhner, 2015), such as the “evolutionary approach”, “constructive approach” or the “pruning approach” (which the method we have chosen to use). According to this approach, one begins with an oversized network. In the course of training, it may become clear that particular parameters are not being utilized (e.g., some connection weights go to zero or near-zero); these are then eliminated. While this approach can result in very effective networks, a downside is that this process can be computationally demanding. Given the 72 processor computer cluster that our lab owns, and that will be used to run these models, this is not a major consideration. If the pruning approach fails to produce adequate results, other model-construction approaches can be used: this is necessarily a trial-and-error process.

1.-57. (canceled)

58. A method, comprising:
obtaining electroencephalogram (EEG) signals from a patient;
determining one or more EEG metrics based on the EEG signals, the one or more EEG metrics including a power law exponent of activity at one or more brain locations of the patient; and
identifying the patient as a responder of a psychoactive agent using a model configured to receive the one or more EEG metrics and predict a clinical treatment outcome of the patient based on the one or more EEG metrics.
